(19) United States
(12) Patent Application Publication
(10) Pub. No.: US 2024/0367077 A1
(43) Pub. Date: Nov. 7, 2024

(54) DEPTH FILTER MEDIA

(71) Applicant: MERCK PATENT GMBH, Darmstadt (DE)

(72) Inventors: Axel Delp, FRAENKISCH-CRUMBACH (DE); Michael Schulte, BISCHOFSHEIM (DE); John Paul Amara, Reading, MA (US); Darcy L. Chen, Brighton, MA (US); Michael James Susienka, Sutton, MA (US)

(21) Appl. No.: 18/291,526

(22) PCT Filed: Aug. 25, 2022
(86) PCT No.: PCT/EP2022/073716

§ 371 Date: Jan. 23, 2024

Related U.S. Application Data
(60) Provisional application No. 63/237,680, filed on Aug. 27, 2021.

(51) Int. Cl. B01D 39/16; B01J 20/26; B01J 20/28; B01J 20/30; C07K 1/34

(52) U.S. Cl. CPC B01D 39/1661; B01J 20/261; B01J 20/267; B01J 20/28004; B01J 20/28026; B01J 20/2803; B01J 20/3007; B01J 20/3042; B01J 20/3078; C07K 1/34; B01D 2239/0407; B01D 2239/08; B01D 2239/10; B01D 2239/1241; B01D 2239/1291

(57) ABSTRACT

Depth filtration media for the harvest clarification of a feed stream from cell cultures containing a therapeutic biomol-ecule. The depth filtration media is comprised of a sintered mixture of a polymeric adsorbent and a thermoplastic binder and possess ultra-low extractables and do not require pre-flushing prior to use. Further, the materials of construction used for the sintered depth filter media demonstrate a high stability to gamma radiation and are compatible with gamma-based pre-use sterilization processes.

### CROSS REFERENCE TO RELATED APPLICATIONS

This application claims the benefit of priority of U.S. Provisional Patent Application No. 63/237,680, filed Aug. 27, 2021, the entire contents of which are hereby incorporated by reference in its entirety.

### BACKGROUND OF THE INVENTION

Current biopharmaceutical production processes employ a series of filtration and chromatographic purification steps in order to isolate and purify the therapeutic biological product from a mammalian cell culture suspension. These therapeutic biological products may include monoclonal antibodies, enzymes, antibody fragments, proteins, fusion proteins, among others. The series of filtration and chromatographic purification steps that are used in the isolation and purification of therapeutic biological products is commonly called a downstream purification process (DSP). The first step of a DSP is often a clarification step that is intended to remove whole cells, cellular debris, and colloidal material from the feedstream containing the therapeutic biological product. The clarification step employed in a DSP may include centrifugation, microfiltration (either by tangential flow filtration or normal flow filtration), or more recently, depth filtration.

Conventional depth filters employed in DSP clarification processes are often based on wet-laid mixtures of cellulose, diatomaceous earth, and wet-strength binder resins. Other conventional depth filters employed in DSP clarification processes are based on wet-laid mixtures of synthetic fibers, silica gel filter aids, and wet-strength binder resins. Depth filters of the types described above may also include synthetic nonwoven fiber layers that are intended for the clarification of pre-treated cell culture feed streams by flocculation or other means, or for the removal of whole cells and cellular debris. Selected examples of conventional depth filters of this type include MILLISTAK+®, MILLISTAK+® HC, MILLISTAK+® HC Pro, or CLARISOLVE® depth filters, all commercially available from EMD Millipore Corporation (Burlington, MA).

Conventional depth filters that are manufactured by means of a wet-laid production process typically require extensive pre-use flushing with high purity water in order to reduce organic and inorganic extractables.

There are various sources for the organic and inorganic extractables and these are typically introduced into the depth filter media during the wet-laid production process. Naturally-derived materials of construction, such as cellulose and diatomaceous earth, are also likely sources for certain organic and inorganic extractables components. In a DSP process, the depth filter extractables may contaminate the feedstream containing the therapeutic biological product and these extractables must be removed from the depth filter prior to use in a DSP filtration process.

Yavorsky (U.S. Pat. No. 7,673,757; the '757 patent) discloses filter media for removal of biological contaminants from process liquids. The filters of Yavorsky are made of an adsorbent material fused with a water-insoluble thermoplastic binder. The filters of Yavorsky are described as having reduced extraneous contaminants (total organic carbon: TOC) that require removal with preflushing prior to use as compared to cellulose containing filters. However, the present inventors have found that the filters of the '757 patent still have unacceptably high levels of organic and inorganic contaminants requiring preflushing and, further, may not have the requisite stability to gamma irradiation, used for filter sterilization, needed in the bioprocessing field.

There is a current need for improved depth filtration media with reduced extractables for DSP filtration and clarification applications. Furthermore, there is a current need for improved depth filtration media that are compatible with gamma irradiation-based filter sterilization processes.

### SUMMARY OF THE INVENTION

Herein, we report the development of a novel and non-obvious all-synthetic depth filtration media that comprises a thermally fused mixture of a polyethylene binder and high surface area synthetic filter aid. The all-synthetic depth filtration media of the current invention demonstrates significantly reduced organic and inorganic extractables over prior art depth filters. The all-synthetic depth filtration media of the current invention is prepared by means of a thermal process to fuse the polyethylene binder to the high surface area synthetic filter aid. This process forms a robust filter sheet containing the binder and high surface area filter aid and eliminates the need for a wet-laid filter media production process. Wet-laid filter production processes are a likely source for increased organic and inorganic extractables in conventional depth filtration media. In some embodiments of the present invention, wet-laid filter production processes are specifically excluded as a means of producing the filters of the present invention. Furthermore, the thermal process employed in the present invention also eliminates the need for the wet-strength binder resins that are currently used in conventional depth filtration media. Wet-strength binder resins are another likely source for increased organic extractables in conventional depth filtration media. In one aspect of the present invention, the use of wet-strength binder resins in the depth filter media of the present invention is specifically excluded as an embodiment or aspect of the depth filter of the present invention.

Importantly, the new depth media of the present invention also provides reduced organic and inorganic contamination after exposure to gamma irradiation used for sterilization. This is in sharp contrast to prior art filter media that “shed” increased amounts of contaminants after gamma irradiation, thereby requiring further increases in pre-flushing times and water volumes.

The high surface area filter aids that are used in the construction of the depth filtration media of the present invention serve to remove whole cells, submicron particles, colloidal matter, soluble impurities (HCP and DNA) and cellular debris by adsorption mechanisms including electrostatic and hydrophobic interactions. Such species may not otherwise be trapped by particle sieving. The use of depth filters comprising filter aids for these purposes has been previously reported (Nguyen, et al., Biotechnol. J. 2018, 1700771).

In a preferred embodiment, the synthetic sorbent of the present invention is polystyrene. Examples of suitable polystyrenes known to one of skill in the art include, but are not limited to, one or more of: PrAOH (polystyrene crosslinked with divinylbenzene: anion, hydroxide form; Purolite Corp., King of Prussia, PA), PrCH (polystyrene crosslinked with divinylbenzene: cation, hydrogen form; Purolite Corp, King of Prussia, PA), MB1/1 h (polystyrene crosslinked with divinylbenzene: cation/anion hydrogen form; Purolite Corp., King of Prussia, PA), polystyrene adsorbent (Medapore P787 (>63 μm), Merck KGAa, Darmstadt, Germany), poly(4-vinylbenzene) 2% cross-linked with divinylbenzene (MilliporeSigma, Burlington, MA), poly(4-vinylpyridine-co-ethylvinylbenzene) 25% cross-linked with divinylbenzene (MilliporeSigma, Burlington, MA). Additional adsorbants may include ion exchange resins such as Amberlite™, Amberlite™ XAD™, and AmberChrom™ ion exchange resins (DuPont, Wilmington, DE). Suitable high surface area filter aids for the present invention may also include one or more of various chromatography resins including ESHMUNO® ion exchange chromatography resins and FRACTOGEL® ion exchange chromatography resins, both available from EMD Millipore (Burlington, MA), among others. One skilled in the art will recognize that suitable synthetic high surface area filter aids may be selected provided the filter aid demonstrates a sufficient thermal stability in order to survive the sintering process employed in depth filter media sheet formation. In another aspect of the present invention, diatomaceous earth, silica, porous glass, zeolites, activated carbon, non-synthetic chromatography media and cellulose-based materials, alone or in combination, are specifically excluded as embodiments or aspects of the depth filter of the present invention.

The all-synthetic depth filtration media of the present invention is also comprised of materials of construction that demonstrate a high stability to gamma radiation. As a result, the all-synthetic depth filtration media of the present invention may be sterilized by means of gamma radiation or other radiation sterilization modalities such as X-ray and electron beam without any negative impact on depth filtration performance in downstream purification processes (DSP) clarification applications. Furthermore, the high gamma stability of the materials of construction of the all-synthetic depth filtration media of the present invention, unlike prior art filter media, provides for very low organic and inorganic extractables even after sterilization by means of gamma irradiation.

Thus, the all synthetic depth filter of the present invention provides for a depth filter that is virtually free of organic and inorganic extractables, even after gamma irradiation, and requires very little to no pre-flushing prior to use. It is well known that the polymeric materials used in conventional depth filtration media, such as cellulose and polyacrylic fibers, can experience bond rupturing in the presence of ionizing radiation sources such as gamma rays, X-rays, and electron beams. These molecular changes often result in chain scission, recombination, and/or cross-linking, which can alter the mechanical and/or chemical properties of the original material causing it to be “shed” into the process fluid as extraneous extractables. The compatibility of various polymers to sterilizing doses of gamma radiation has been previously evaluated. See Kawamura, Y. “Effects of Gamma Irradiation on Polyethylene, Polypropylene, and Polystyrene” In: Irradiation of Food and Packaging. Chapter 16 ACS Symposium Series. American Chemical Society, Washington, D.C., 2004; 262-276. In another aspect of the present invention, no pre-flushing prior to use is needed exclusive of filling the filtration device containing the filter media with water or the solution intended for filtration. In yet another aspect of the present invention, pre-flushing the depth filter of the present invention, exclusive of enough liquid to wet the filter, is explicitly excluded as an embodiment of the present invention.

In one aspect, the present invention contemplates a depth filter comprising; a) a sintered depth filter media comprising a thermally fused mixture of polyethylene binder, and b) one or more adsorbents selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene. In another aspect of the present invention, the present invention contemplates that the depth filter media is significantly homogeneous. In another aspect of the present invention, the present invention contemplates that the depth filter is from about 1 mm—about 4 mm thick or from about 2 mm—about 3 mm thick.

In another aspect, the present invention contemplates that wet-strength binder resins are specifically excluded.

In another aspect, the present invention contemplates that ratio of polyethylene to adsorbent is from about 1:10 to about 10:1; from about 1:5 to about 5:1; from about 1:2 to about 2:1; or said ratio of polyethylene to adsorbent is about 1:1.

In another aspect, the present invention contemplates that the size of said polystyrene adsorbent is from about 10 μm to about 120 μm in diameter; from about 30 μm to 100 μm in diameter; from about 50 μm to 80 μm in diameter; from about 60 μm to 70 μm in diameter or is about 63 μm in diameter.

In another aspect, the present invention contemplates a method of making a sintered depth filter media, the method comprising mixing a polyethylene binder with an adsorbent selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene to make a mixture, spreading the mixture to a substantially uniform thickness and heating the mixture at about 165° C. for about 60 minutes.

In another aspect, the present invention contemplates a method of clarifying a feedstream, the feed stream containing a target protein, the method comprising: a) providing: a depth filter comprising a sintered depth filter media, said depth filter media comprising a polyethylene binder with an adsorbent selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene; b) contacting the depth filter with the feed stream such that the target protein passes through the depth filter and is retained in the permeate and contaminants are adsorbed by the depth filter, such that the concentration of target protein to contaminant in the permeate is increased by at least 20% as compared to the ratio in the feed stream.

In another aspect, the present invention contemplates that the concentration of target protein to contaminant in the permeate is increased by at least 50% as compared to the ratio in the feed stream.

In another aspect of the present invention, the present invention contemplates that the concentration of target protein to contaminant in the permeate is increased by at least 100% as compared to the ratio in the feed stream.

### BRIEF DESCRIPTION OF THE FIGURES

FIG. 1 shows a photograph of the sintered depth filter media of the present invention. The sintered depth filter media was prepared as described in Example 3, Example ID 3-2. A photograph of a cellulose-based benchmark sample, MILLISTAK+® CE25 filter media (MilliporeSigma, Burlington, Ma) is shown for comparison.

FIGS. 2(A-D) show cross-section SEM micrographs of the sintered depth filter media of the present invention. The sintered depth filter media was prepared as described in Example 3, Example ID 3-2. The SEM micrographs are provided at 100× (A & B) and 500× (C & D) magnification. The SEM micrographs show the large >63 μm polystyrene beads are entrapped by the much smaller fused polyethylene particles.

FIG. 3 shows TOC flush out curve for a 23 cm² minicap filter device containing a single layer of the sintered depth filter media of the present invention and as described in Example 3.

FIG. 4 shows TOC flush out curves for 23 cm² minicap filter devices each containing a single layer of the sintered depth filter media of the present invention as described in Example 3 pre- and post-gamma irradiation.

FIG. 5 shows metallic extractables data for a 23 cm² minicap filter device containing a single layer of the sintered depth filter media of the present invention and as described in Example 3.

FIG. 6 shows metallic extractables data for 23 cm² minicap filter devices each containing a single layer of the sintered depth filter media of the present invention and as described in Example 3 for selected extractables with and without gamma irradiation.

FIG. 7 shows filter resistance profiles for application tests conducted using the sintered depth filter media for the clarification of CHO cell culture harvest. The details of this application/experiment are provided in Example 6.

FIG. 8 shows filter resistance profiles for application tests conducted using the sintered depth filter media for the clarification of a Peptone HY-SOY® T (Milliporesigma, Burlington, MA) model feed stream. The details of this application/experiment are provided in Example 7.

FIG. 9 shows a prior art comparison of the conductivity of water filtrate from the '757 patent of various prior art filter media during flushing.

FIG. 10 shows the conductivity of the water filtrate during flushing of the filter media of the present invention as compared to two prior art media before and after gamma irradiation.

FIG. 11 shows a conductivity increase of the water filtrate during flushing of prior art CE25 media after gamma irradiation.

FIG. 12 shows a conductivity increase of the water filtrate during flushing of prior art DE40 media after gamma irradiation

FIG. 13 shows no increase in conductivity of the water filtrate during flushing of the media of the present invention after gamma irradiation.

FIG. 14 shows prophetically the reduced conductivity of the water filtrate during flushing of the filter media of the present invention over various, known prior art media both with and without exposure to gamma irradiation.

### DETAILED DESCRIPTION OF THE INVENTION

### Definitions

Unless defined otherwise, all technical and scientific terms used herein have the meaning commonly understood by a person skilled in the art to which this invention belongs. The following reference will provide one of skill with a general definition of many of the terms used in this invention: Process Scale Bioseparations for the Biopharmaceutical Industry, edited by Abhinav A. Shukla, Mark R. Etzel, and Shishir Gadam. As used herein, the following terms have the meanings ascribed to them unless specified otherwise.

When introducing elements of the present disclosure or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03 (Manual of Patent Examining Procedure; United States Patent and Trademark Office). Any claims using the transitional phrase “consisting essentially of” will be understood as reciting only essential elements of the invention and any other elements recited in dependent claims are understood to be non-essential to the invention recited in the claim from which they depend.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about” whether or not explicitly indicated. The term “about” generally refers to a range of numbers that one would consider equivalent to the recited value (i.e., having the same function or result). In many instances, the term “about” may include numbers that are rounded to the nearest significant figure.

Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Moreover, all ranges disclosed herein are to be understood to encompass all sub ranges subsumed therein. In other words, any ranges of numbers are considered to include all numbers within the range as if they were explicitly recited.

Before describing the present invention in further detail, a number of terms will be defined. Use of these terms does not limit the scope of the invention but only serve to facilitate the description of the invention.

All publications, patents and patent applications cited herein, whether supra or infra, are hereby incorporated by reference in their entirety to the same extent as if each individual publication, patent or patent application was specifically and individually indicated to be incorporated by reference. All publications, patents and patent applications cited herein are also representative of what one of ordinary skill in the art would understand with regard to the field of the invention at the time of the invention.

The term “bubble point pore size” or “BP” is the pore size of the largest pore in the filter medium.

As used herein the phrase “cell culture” includes cells, cell debris and colloidal particles, biomolecule of interest, HCP, and DNA.

The terms “Chinese hamster ovary cell protein” and “CHOP” as used interchangeably herein, refer to a mixture of host cell proteins (“HCP”) derived from a Chinese hamster ovary (“CHO”) cell culture. The HCP or CHOP is generally present as an impurity in a cell culture medium or lysate (e.g., a harvested cell culture fluid containing a protein or polypeptide of interest (e.g., an antibody or immunoadhesion expressed in a CHO cell). Generally, the amount of CHOP present in a mixture comprising a protein of interest provides a measure of the degree of purity for the protein of interest. Typically, the amount of CHOP in a protein mixture is expressed in parts per million relative to the amount of the protein of interest in the mixture.

The term “clarification step” or simply “clarification”, as used herein, generally refers to one or more steps used initially in the purification of biomolecules. The clarification step generally comprises removal of cells and/or cellular debris using one or more steps including any of the following alone or various combinations thereof, e.g., centrifugation and depth filtration, tangential flow filtration, microfiltration, precipitation, flocculation and settling. In some embodiments, the present invention provides an improvement over the conventional clarification step commonly used in various purification schemes. The clarification step generally involves the removal of one or more undesirable entities and is typically performed prior to a step involving capture of the desired target molecule. Another aspect of clarification is the removal of soluble and insoluble components in a sample which may later on result in the fouling of a sterile filter in a purification process, thereby making the overall purification process more economical. The clarification step often includes a primary clarification step(s) upstream and a secondary clarification downstream. The clarification of cell culture harvests and high-solids feedstocks from large harvest volumes from modern production batch bioreactors (<25,000 L) and high cell densities often require primary, as well as secondary clarification steps prior to any subsequent chromatography operations and the like.

The terms “coarse filtration” or “coarse/medium filtration,” as used herein, generally refer to the removal of mostly whole cells and some cellular debris in the purification of biomolecules.

The term “fine filtration,” as used herein, generally refers to the removal of mostly cellular debris, colloidal particles and soluble impurities such as HCP, DNA, endotoxins, viruses and lipids in the purification of biomolecules.

Filter throughput values are generally expressed in terms of “liters/square meter” or “L/m²” though for equivalent comparisons, “column volume” or “CV” is used to account for large differences of thickness between samples.

The terms “contaminant,” “impurity,” and “debris” are used interchangeably herein and refer to any foreign or objectionable material, including a biological macromolecule such as a DNA, an RNA, one or more host cell proteins (HCPs or CHOPs), endotoxins, viruses, lipids and one or more additives which may be present in a sample containing a protein or polypeptide of interest (e.g., an antibody) being separated from one or more of the foreign or objectionable molecules using a depth filter according to the present invention.

It is understood that where the host cell is another mammalian, non-mammalian, or bacterial cell type, for example, E. coli, yeast cell, insect or plant, HCP refers to the proteins, other than target proteins, found in a lysate of the host cell.

The term “monoclonal antibody” or “mAb” as used herein refers to an antibody obtained from a population of substantially homogeneous antibodies, i.e., the individual antibodies comprising the population are identical except for possible naturally occurring mutations that may be present in minor amounts.

The term “mean flow pore size” or “MFP” as used herein is the pore diameter at a pressure drop at which the flow through a wetted filter medium is 50% of the flow through the dry filter medium.

As used herein the term “organic extractable(s)” refers to contaminants that in the presence of water or other aqueous solutions used during flushing, can potentially migrate or be extracted from materials used to make filter media or membranes, such as porous depth filter media. These contaminants may also include the materials of construction themselves which could potentially shed from the filter during use thereby requiring pre-flushing of the filter prior to use of the filter to remove said organic extractable(s).

The term “total organic extractable(s)” and “TOC” refers to the measurement of organic molecules present in an aqueous solution such as water and measured as carbon content. Analytical techniques used to measure TOC typically involve oxidation of all organic molecules in solution to carbon dioxide, measuring the resultant CO₂ concentration, and correlating this response to a known carbon concentration.

The term “inorganic extractable” refers to trace metallic species, including heavy metals that may be extracted from the filter into the process fluid. These metallic species may be measured by analytical techniques such as inductively coupled plasma optical emission spectrometry (ICP-OES), ICP mass spectrometry (ICP-MS), and graphite furnace atomic absorption spectrometer (GFAAS) techniques.

“Pre-flush” is defined herein as flushing a filter prior to use, usually with sterile water, to remove organic and inorganic extractables from the filter prior to use.

The term “parts per million” or “ppm” are used interchangeably herein.

Pore size ratings are usually given as a nominal value. In some cases, manufacturers provide a mean flow pore (MFP) size or a bubble point (BP) pore size. Both the MFP and BP can be measured using a capillary flow porometer.

The terms “target molecule,” “target biomolecule,” “desired target molecule” and “desired target biomolecule” are used interchangeably herein and generally refer to a polypeptide or product of interest (e.g., a monoclonal antibody) which is desired to be purified or separated from one or more undesirable entities, e.g., one or more impurities, which may be present in a sample containing the polypeptide or product of interest.

As used herein the term “throughput” means the volume filtered through a filter divided by the frontal area of the filter. The throughput is expressed in terms of L of fluid filtered/m² of filter area.

As used herein the term “dirt holding capacity” is equivalent to filter throughput of a given cell culture fluid, either from direct harvest or previously clarified. Higher throughput represents higher dirt holding capacity.

As used herein and as understood to one of ordinary skill in the art, “wet-strength binder resin” is a resin that allows for the formation of filter sheets by binding fibers and/or particles together, increases the wet strength of the filter and, depending on the binder used, can impart unique charge properties. Examples of wet-strength resins are resins comprising synthetic polymers of urea or melamine-formaldehyde based polymers, polyaminopolyamide-epichlorohydrin (PAE) polymers and glyoxalated polyacrylamide (GPAM) resins.

### Sintered Filters of the Present Invention

The filters of the present invention are referred to herein as “sintered filters.” The term “sintered” is known to one of skill in the art to refer to an item produced by or subjected to sintering, which is the process of coalescing a powdered material into a solid or porous mass by means of heating without liquefaction. The filters of the present invention comprise (or “consist essentially of” or “consist of”) a high surface area synthetic filter aid bound by a polyethylene binder through thermal fusion. High surface area synthetic filter aids are defined as having a BET surface area of greater than 10 m²/g. In some cases, the BET surface area of the synthetic filter aid may be greater than 100 m²/g. BET measures surface area based on gas adsorption (Adsorption of Gases in Multimolecular Layers, Brunauer, Emmett, Teller, J. Am. Chem. Soc., February 1938, vol. 60, 309). Alternate binders may also include thermoplastic powders including nylon 6 powder (PA6, MilliporeSigma, Burlington, MA), and nylon 12 powder (PA12, MilliporeSigma, Burlington, MA). Alternate binders may also include acrylic staple fibers, Fybrel® synthetic wood pulp, Short Stuff® fibrillated HDPE (Minifibers, Inc.).

The filter of the present invention, in a preferred aspect, is “homogeneous” or “significantly homogeneous,” meaning that the ratio of synthetic filter aid to binder being the same or substantially the same over the entire filter and that the composition with regard to the ratio of synthetic filter aid to binder varies only within manufacturing tolerances.

The filter retention characteristics of the depth filter of the pending invention can vary depending upon the polymers, mixtures and ratios selected. Generally, the depth filter of the present invention will show an increased permeability and decreased retention of fine particles with an increased loading of the high surface area synthetic filter aid. Conversely, a decreased loading of the high surface area synthetic filter aid in the sintered depth filter of the present invention will show a decreased permeability and increased retention of fine particles. In addition, one skilled in the art will consider the trade-off between filter permeability and filter retention of the sintered filter media sheet in light of a reduction in the mechanical integrity of the sintered filter sheet at high loading values of the high surface area synthetic filter aid >70 wt %.

The permeability of the depth filter sheet may be evaluated by determining the pressure drop in PSI of a filtration device containing the depth filter sheet at various water flux rates in LMH. A higher pressure drop is indicative of lower permeability of the filter sheet.

The process flux is expressed in terms of the volumetric flow rate of fluid being filtered divided by the frontal area of the filtration device in m². The process flux is expressed in terms of L/m²/hr (LMH). Typical process flux for biopharmaceutical depth filtration processes range between 75 and 300 LMH. The pressure drops for the sintered filter media of the present invention are less than 1.1 psi at a flux of 574 LMH.

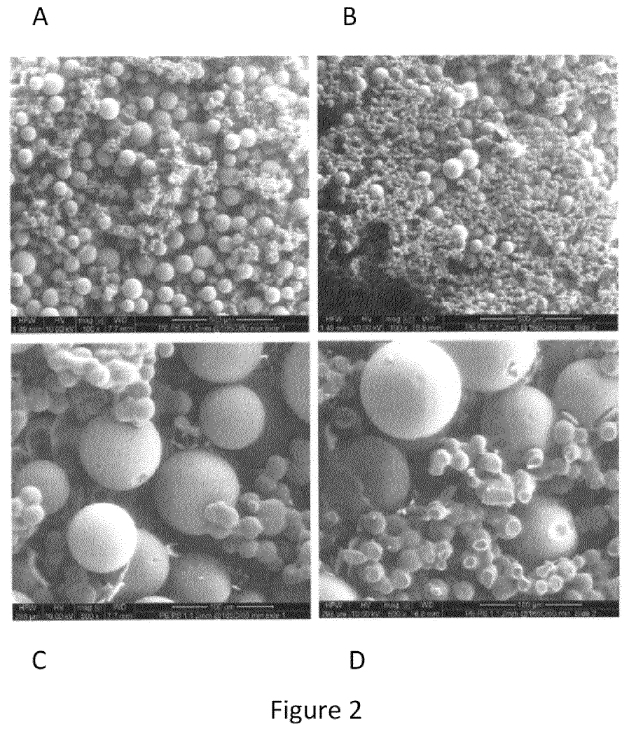

As mentioned, supra, the filters of the present invention are produced by thermal fusion. Examples of the thermal fusion process are given in the Exemplification section, below. Thermal fusion, sometimes called heat fusion, is a process known in the art that is used to join, for example, thermoplastics together. Instead of using, for example, glue, solder or a mechanical connection and gasket, heat fusion actually turns the two pieces of plastic into one solid piece by softening and or melting one or more of the thermoplastics enough to bind the other thermoplastic(s) when cooled. The fused sintered filters of the present invention maintain the distinction between the components, i.e., the synthetic filter aid and binder are still visible under magnification as components. See, FIG. 2.

The synthetic sintered depth filters of the present invention are not limited by thickness but, in preferred embodiment, may range in thickness from 0.1 mm-5.0 mm, 0.5 mm-4.0 mm, 1.0 mm-4.0 mm, 1.0 mm-3.0 mm and 2.0 mm-3.0 mm.

The synthetic sintered depth filters of the present invention are not limited by the ratio of binder to synthetic sorbent filter aid. In one aspect, the ratio may be from 1:10-10:1 binder: synthetic sorbent filter aid, 1:5-5:1 binder: synthetic sorbent filter aid, 1:2-2:1 binder: synthetic sorbent filter aid or about 1:1 binder: synthetic sorbent filter aid. One of ordinary skill in the art will be able to determine a suitable ratio for any particular use without undue experimentation with the guidance provided by this specification.

The synthetic sorbent filter aid of the present invention is typically spherical or slightly oblong and has a diameter of about 10 μm to about 120 μm in diameter, about 30 μm to about 100 μm in diameter, about 50 μm to about 80 μm in diameter, about 60 μm to about 70 μm in diameter or about 63 μm in diameter. One of ordinary skill in the art will be able to determine an optimal size for any particular use without undue experimentation with the guidance provided by this specification.

### Methods of Making the Sintered Depth Filters of the Present Invention

The present invention also provides methods of making the sintered depth filters of the present invention. Preferred methods are provided in the exemplification section, infra. In one aspect, a polyethylene binder and polystyrene sorbent are mixed at the desired ratio. The mixture is distributed evenly onto large metal baking sheet and leveled with a drawbar to 2 mm. The baking sheet is then baked for about 60 minutes at about 165° C. After cooling, the sheets of filter material were cut to desired size. One of skill in the art will be able to determine times and temperatures for making the synthetic depth filters of the present invention that utilize a different binder or binders or different sorbent or sorbents, without undue experimentation with the guidance provided by the present invention. Likewise, one of skill in the art will be able to determine times and temperatures for making the synthetic depth filters of the present invention that are thicker or thinner than 2 mm, without undue experimentation with the guidance provided by the present invention

Other methods of making the depth filter may employ automated machinery that allow for the manufacture of large quantities of filter media in an efficient manner.

### Methods of Use of the Sintered Depth Filters of the Present Invention

The present invention contemplates methods of use for the synthetic sintered depth filters of the present invention. For example, the sintered depth filters of the present invention may be used for filtering cell culture media (i.e., a feed stream) in which a bioproduct has been produced. Depending on the stage of processing of the feed stream, filters of differing pore size, porosity, and flow grade may be used. One of skill in the art will be able to determine the correct pore size, porosity and flow grade for a particular use with the aid of the teachings of this specification. In one aspect, the synthetic depth filter of the present invention may be used to clarify a feed stream prior to further downstream processing. A clarification step would remove, for example, cell culture debris such as whole cells, ruptured cells, large host cell proteins (HCP), and other contaminants, etc., while allowing the target proteins to pass through the filter. In one embodiment, the target protein is a monoclonal antibody, humanized monoclonal antibody, CAR-T cell produced antibody, etc. The target protein may also be other genetically engineered or naturally occurring proteins, for example, produced by a cell transfected with an expression vector engineered for expression of the desired target protein or naturally expressed by a given cell type.

The result of passing a cell culture feed stream through a depth filter of the present invention is to increase the relative proportion of target protein to the contaminant(s) in the permeate (i.e., the feed stream that passes through the filter). In this context, the contaminants may include whole cells, cellular debris, and colloidal particles. In one aspect, the concentration of target protein to contaminant(s) is increased by at least 20%, at least 30%, at least 40%, at least 50%, at least 60%, at least 70%, at least 80%, at least 90%, at least 100%, at least 200%, at least 500% and at least 1000%, as compared to the feed stream. In one aspect, the concentration of target protein to contaminant(s) is increased by up to 20%, up to 30%, up to 40%, up to 50%, up to 60%, up to 70%, up to 80%, up to 90%, up to 100%, up to 200%, up to 500% and up to 1000% as compared to the feed stream. In one aspect, the concentration of target protein to contaminant(s) is increased by from 10%-1000% and 50%-500%.

The present invention contemplates that the depth filter of the present invention is not pre-flushed prior to use for filtering a feed stream. In one aspect, pre-flushing of the depth filter of the present invention prior to filtering the feed stream is specifically excluded.

Exemplification

### Example 1

Preparation of sintered depth filter media of the present invention. Polyethylene binder, PE (MIPELON™ XM-221u polyethylene binder, Mitsui Chemicals America, Inc.) and polystyrene adsorbant, PS (Medapore P787, >63 μm, Merck KGaA, Darmstadt, Germany) material were mixed at the ratio described in Table 1, below. The powder mixture was distributed onto a large metal baking sheet with a PTFE release film. A drawbar was used to evenly distribute the powder mixture at a thickness of 2 mm. The baking sheet was placed into a pre-heated oven at 165° C. for 60 min. The samples were removed from the oven and allowed to cool to room temperature. The sintered depth filter sheets were then cut into 23 cm² discs and incorporated into minicap filter test devices for applications testing. The use of minicap filter test devices in the evaluation of depth filter performance has been previously reported (Lutz, H., et al. Biotechnol. Prog., 2015, 31, 6, 1542-1550; which is exemplary of the knowledge of one of ordinary skill in the art for this subject).

TABLE 1

| Example | PE | | | | Thickness |
|---|---|---|---|---|---|
| ID | binder (g) | PS (g) | Total (g) | Temp (° C.) | (mm) |
| 1-1 | 15 g | 15 g | 30 g | 165 | 2 |
| 1-2 | 15 g | 15 g | 30 g | 165 | 2 |

### Example 2

Preparation of sintered depth filter media of the present invention. Polyethylene binder, PE (MIPELON™ XM-221u polyethylene binder, Mitsui Chemicals America, Inc.) and MICROLITE® ion exchange adsorbants (PrAOH, PrCH, and MBI/IH (PUROLITE® Corporation, Bala Cynwyd, PA) material were mixed at the ratios described in Table 2, below. For examples 2-4, 2-5, and 2-6 (as indicated in Table 2, below), the MICROLITE® ion exchange adsorbants were dried at 125° C. for 3 to 18 hrs. The powder mixture was distributed onto a large metal baking sheet with a PTFE release film. A drawbar was used to evenly distribute the powder mixture at a thickness of 3 mm. The baking sheet was placed into a pre-heated oven at 165° C. for 60 min. The samples were removed from the oven and allowed to cool to room temperature. The sintered depth filter sheets were then cut into 5 cm² discs and incorporated into 5 cm² filter test devices for applications testing.

TABLE 2

| | | Microlite ® | Microlite ® | | | | | |
|---|---|---|---|---|---|---|---|---|
| | | IX | resin dried | | | | | |
| Example | Binder | resin | at 125° C., | Total | PE:IX | Temp | Thickness | |
| ID | (g) | (g) | % solids | (g) | ratio | (° C.) | (mm) | Comments and Description |
| 2-1 | PE, 15 g | PrAOH, | No, 29% | 30 g | 1:1 | 165 | 3 | Resulting filter sheet |
| | | 51.7 g | | | | | | cracked. |
| 2-2 | PE, 15 g | PrCH, | No, 49% | 30 g | 1:1 | 165 | 3 | Resulting filter sheet |
| | | 30.6 g | | | | | | cracked. |
| 2-3 | PE, 15 g | MB1/1H, | No, 47% | 30 g | 1:1 | 165 | 3 | Resulting filter sheet |
| | | 31.9 g | | | | | | cracked. |
| 2-4 | PE, 15 g | PrAOH, | Yes | 30 g | 1:1 | 165 | 3 | Filter sheet intact. Anion |
| | | 51.7 g | | | | | | exchange - hydroxide |
| | | | | | | | | form. Microlite ® PrCH is a |
| | | | | | | | | polystyrene crosslinked |
| | | | | | | | | with divinylbenzene with a |
| | | | | | | | | quaternary ammonium |
| | | | | | | | | functional group. |
| 2-5 | PE, 15 g | PrCH, | Yes | 30 g | 1:1 | 165 | 3 | Filter sheet intact. Cation |
| | | 15 g | | | | | | exchange - acid form. |
| | | | | | | | | Microlite ® PrCH is a |
| | | | | | | | | polystyrene crosslinked |
| | | | | | | | | with divinylbenzene with a |
| | | | | | | | | sulfonic acid functional |
| | | | | | | | | group. |
| 2-6 | PE, 15 g | MB1/1H, | Yes | 30 g | 1:1 | 165 | 3 | Filter sheet intact. |
| | | 15 g | | | | | | Mixed bed ion exchange - |
| | | | | | | | | Microlite ® MB1/1H is a |
| | | | | | | | | mixture of polystyrenes |
| | | | | | | | | crosslinked with |
| | | | | | | | | divinylbenzene with either |
| | | | | | | | | a quaternary ammonium |
| | | | | | | | | functional group or a |
| | | | | | | | | sulfonic acid functional |
| | | | | | | | | group. |

### Example 3

Preparation of sintered depth filter media of the present invention. Polyethylene binder, PE (MIPELON™ XM-221u polyethylene binder, Mitsui Chemicals America, Inc.) and polystyrene adsorbant, PS (Medapore P787, >63 μm, Merck KGaA, Darmstadt, Germany) material were mixed at the ratio described in Table 3, below. The powder mixture was distributed onto a large metal baking sheet with a PTFE release film. A drawbar was used to evenly distribute the powder mixture at a thickness of 2 mm. The baking sheet was placed into a pre-heated oven at 165° C. for 60 min. The samples were removed from the oven and allowed to cool to room temperature. The sintered depth filter sheets were then cut into 23 cm² discs and incorporated into minicap filter test devices for applications testing. The use of minicap filter test devices in the evaluation of depth filter performance has been previously reported (Lutz, H., et al. Biotechnol. Prag., 2015, 31, 6, 1542-1550; which is exemplary of the knowledge of one of ordinary skill in the art for this subject).

TABLE 3

| | PE | | | | Thick- | |
|---|---|---|---|---|---|---|
| Example | binder | PS | Total | Temp | ness | |
| ID | (g) | (g) | (g) | (° C.) | (mm) | Comments |
| 3-1 | 5 g | 5 g | 10 g | 225 | 2 | Yellowed, powdery |
| 3-2 | 5 g | 5 g | 10 g | 165 | 2 | Filter sheet intact. |
| 3-3 | 3 g | 7 g | 10 g | 165 | 2 | Filter sheet intact. |
| 3-4 | 7 g | 3 g | 10 g | 165 | 2 | Filter sheet intact. |
| 3-5 | 6 g | 4 g | 10 g | 165 | 2 | Filter sheet intact. |
| 3-6 | 4 g | 6 g | 10 g | 165 | 2 | Filter sheet intact. |
| 3-7 | 15 g | 15 g | 30 g | 165 | 2 | Filter sheet intact. |

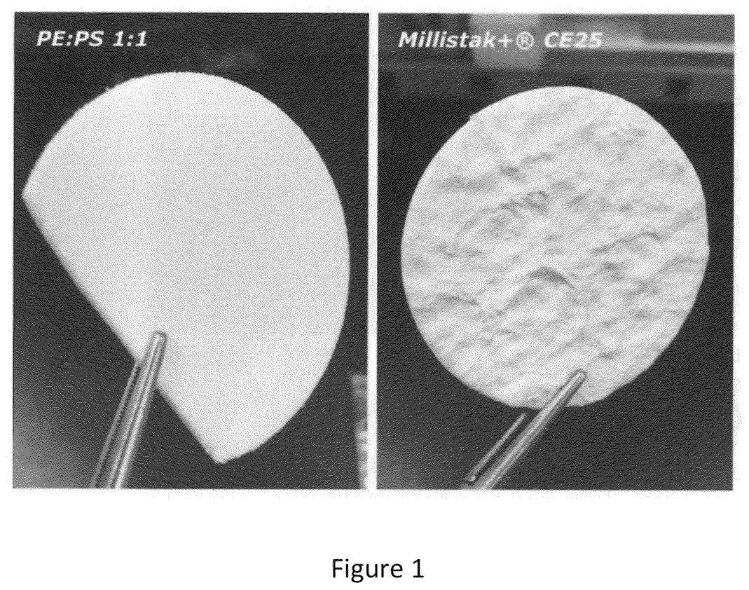

Filter media made according to this example is shown in FIGS. 1 and 2. FIG. 1 shows the sintered depth filter media prepared as in Example ID 3-2 (A). A photograph of a cellulose-based benchmark sample (MILLISTAK+® CE25 filter media) is shown for comparison (B).

In FIG. 2 a cross-section SEM micrographs of the sintered depth filter media of the present invention. The sintered depth filter media shown in FIG. 2 was made according to Example ID 3-2. The SEM micrographs are provided at 100×(A & B) and 500×(C & D) magnification.

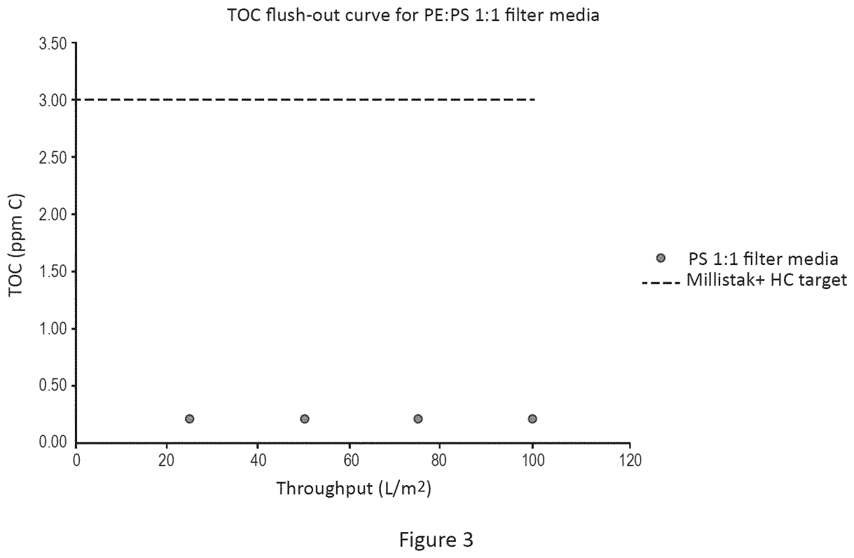

The sintered depth filter media was prepared as described for Example ID 3-7. As shown in FIG. 3, the minicap filter device was flushed with 100 L/m² of Milli-Q (MilliporeSigma, Burlington, MA) water at 600 LMH. TOC extractables samples were collected after each 25 L/m² of flushing. The TOC extractables values recorded for the test device containing the sintered depth filter media of Example ID 3-7 are far below the typical TOC flush out target for the MILLISTAK+® HC (MilliporeSigma, Burlington, MA) (<3 ppm TOC at 100 L/m² flush-out).

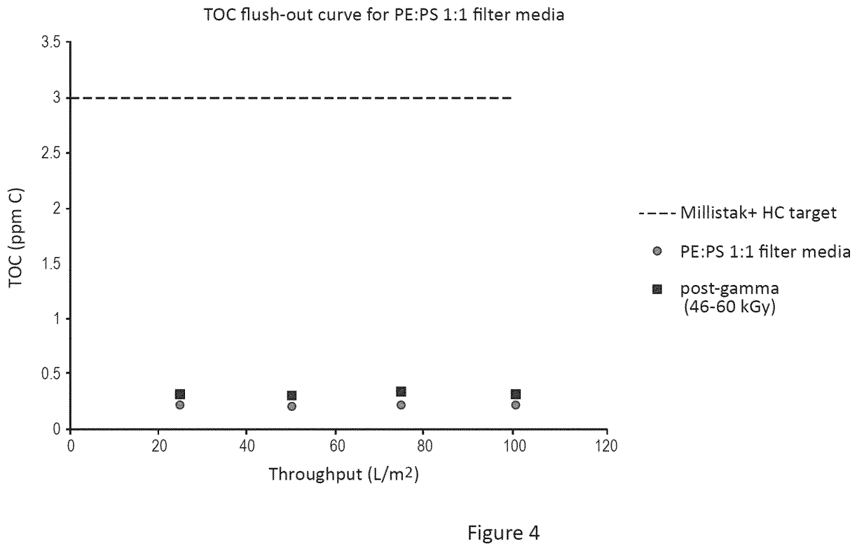

The sintered depth filter media was prepared as described for Example ID 3-7. As shown in FIG. 4, one of the 23 cm² minicap filter devices (squares) was subjected to a 40-60 kGy gamma radiation dose and one was not (circles). Each of the minicap filter devices were flushed with 100 L/m² of Milli-Q water at 600 LMH. TOC extractables samples were collected after each 25 L/m² of flushing. The TOC extractables values recorded for the test devices containing the sintered depth filter media of Example ID 3-7 are far below the typical TOC flush out target for the MILLISTAK+® HC (<3 ppm TOC at 100 L/m² flush-out). No significant increase in TOC extractables was observed for the device that was subjected to 40-60 kGy of gamma radiation. These results show that sintered depth filter media of the present invention would not require pre-use device flushing for typical clarification operations used in biopharmaceutical manufacturing. Furthermore, the materials of construction of the sintered depth filter media demonstrate good stability to gamma radiation

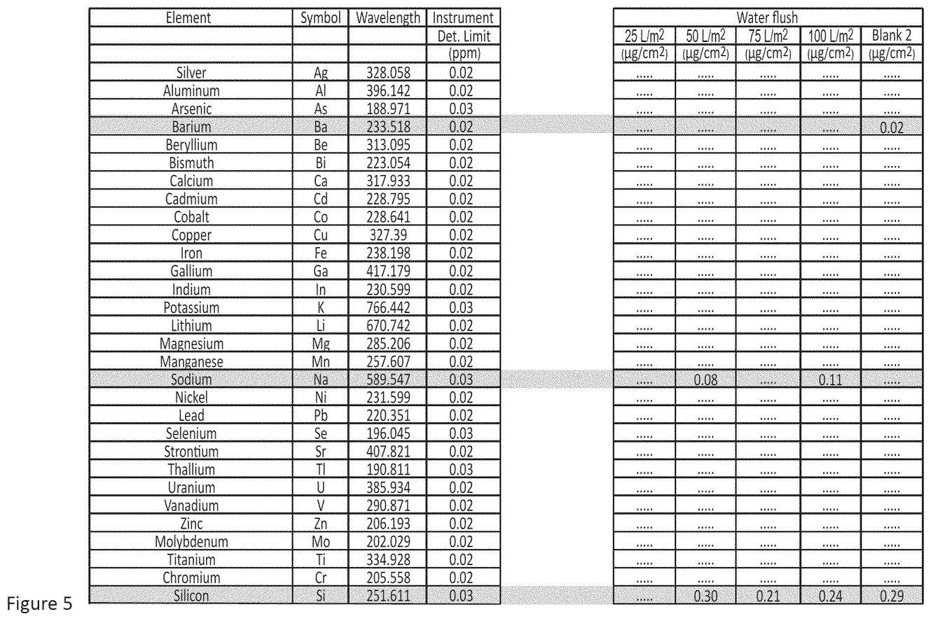

The sintered depth filter media was prepared as described for Example ID 3-7. As shown in FIG. 5, the minicap filter device was flushed with 100 L/m² of Milli-Q water at 600 LMH. Metallic extractables samples were collected after each 25 L/m² of flushing. Metallic extractables were measured by means of inductively coupled plasma optical emission spectroscopy (ICP-OES). The metallic extractables values recorded for test device containing the sintered depth filter media of Example ID 3-7 are below the instrument limit of detection (0.02 ppm) for all metals with the exception of silicon and sodium.

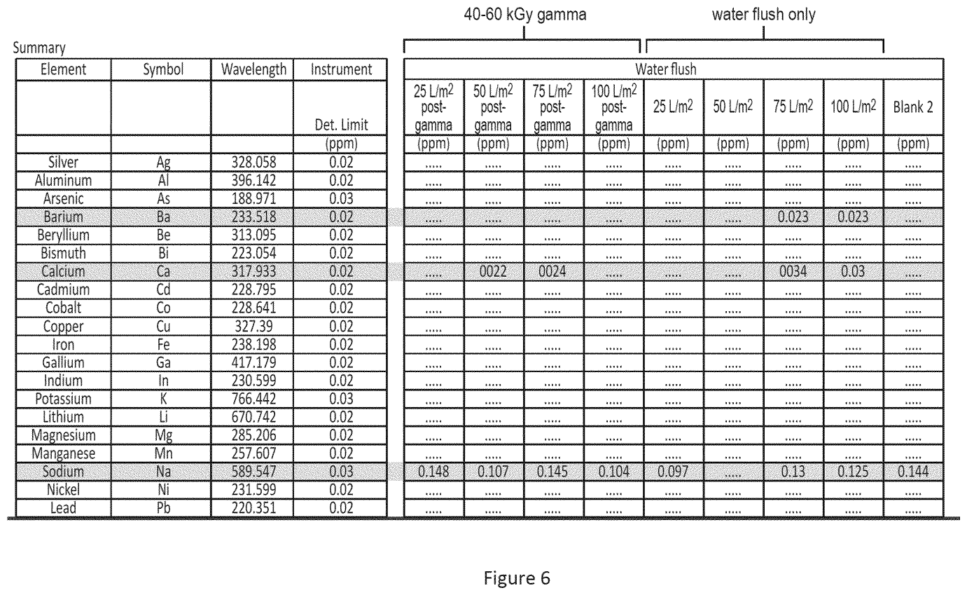

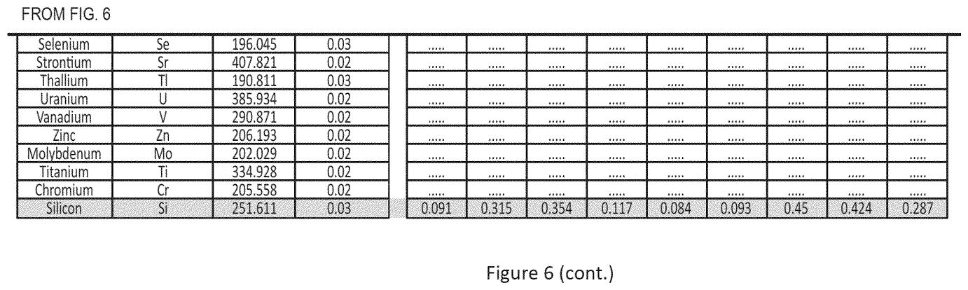

The sintered depth filter media was prepared as described for Example ID 3-7. As shown in FIG. 6, one of the 23 cm² minicap filter devices was subjected to a 40-60 kGy gamma radiation dose. Each of the minicap filter devices were flushed with 100 L/m² of Milli-Q water at 600 LMH. Metallic extractables samples were collected after each 25 L/m² of flushing. Metallic extractables were measured by means of inductively coupled plasma optical emission spectroscopy (ICP-OES). No significant increase in metallic extractables was observed for the device that was subjected to 40-60 kGy of gamma radiation. These results show that sintered depth filter media of the present invention would not require pre-use device flushing for typical clarification operations used in biopharmaceutical manufacturing. Furthermore, the materials of construction of the sintered depth filter media demonstrate excellent stability to gamma radiation.

### Example 4

Preparation of sintered depth filter media of the present invention. Polyethylene binder, PE (MIPELON™ XM-221u polyethylene binder, Mitsui Chemicals America, Inc.) and cross-linked poly(4-vinylpyridine) adsorbants, PVP. The PVP adsorbants included poly(4-vinylpyridine), 2% cross-linked with divinylbenzene, and poly(4-vinylpyridine-co-ethylvinylbenzene), 25% cross-linked with divinylbenzene, both were purchased from MilliporeSigma, St. Louis, MO, USA). These materials were mixed at the ratios described in Table 4, below. The powder mixture was distributed onto a large metal baking sheet with a PTFE release film. A drawbar was used to evenly distribute the powder mixture at a thickness of 2 mm. The baking sheet was placed into a pre-heated oven at 165° C. for 120 min. The samples were removed from the oven and allowed to cool to room temperature.

TABLE 4

| Example | PE binder | | Total | Temp | Thickness | |
|---|---|---|---|---|---|---|
| ID | (g) | PVP resin type, (g) | (g) | (° C.) | (mm) | Comments |
| 4-1 | 15 g | Poly(4-vinylpyridine), | 30 g | 165 | 2 | Filter sheet intact. |
| | | 15 g | | | | |
| 4-2 | 15 g | Poly(4-vinyl-pyridine- | 30 g | 165 | 2 | Filter sheet intact. |
| | | co-ethylvinylbenzene), | | | | |
| | | 15 g | | | | |

### Example 5

Water flow rate testing of the sintered depth filter media after gamma irradiation. 23 cm² minicap test devices containing the sintered depth filter media described in Example 1 were provided. One of the 23 cm² minicap test devices containing the sintered depth filter media of the present invention was subjected to gamma irradiation at a dose of 40-60 kGy. The described minicap devices were attached to a peristaltic pump and deionized water was pumped through the devices and the flow rate was measured by means of a scale and timer. The pressure drop was measured by means of an electronic pressure transducer. The results of the water flow rate testing are shown in Table 5, below. Filtration devices comprising the sintered depth filtration media of the present invention demonstrate sufficiently low pressure drops at a relevant flux for application in harvest clarification unit operations.

TABLE 5

| Example | | Volume | Time | Flux | |
|---|---|---|---|---|---|
| ID | Filter Media | (mL) | (min) | (LMH) | ΔP (PSI) |
| 5-1 | example 1-1 | 110 | 5 | 574 | 1.1 |
| 5-2 | example 1-1 (40-60 | 100 | 5 | 522 | 0.4 |
| | kGy gamma dose) | | | | |

### Example 6

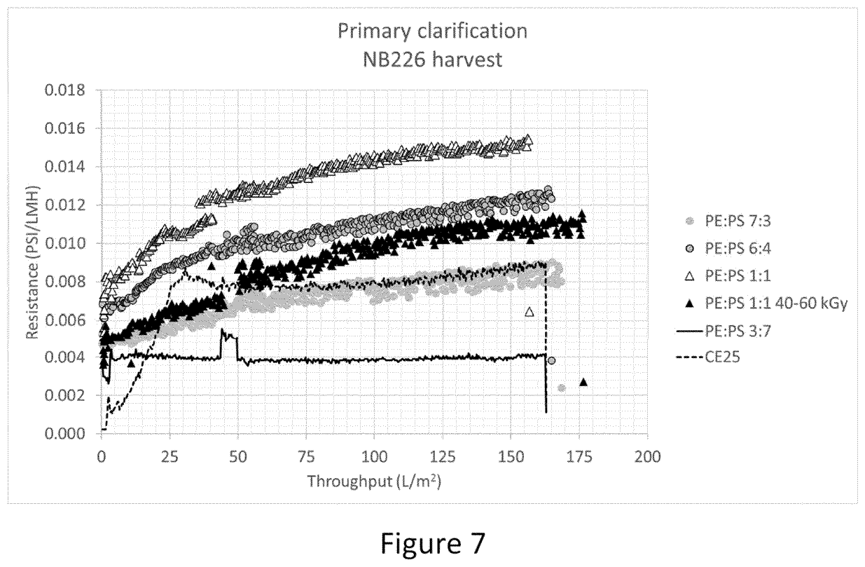

Clarification of CHO cell culture harvest. 23 cm² minicap test devices containing the sintered depth filter media described in Example 3 were provided. One of the 23 cm² minicap test devices containing the sintered depth filter media of the present invention was subjected to gamma irradiation at a dose of 40-60 kGy. The filter test devices were challenged with a CHO cell culture harvest having a total cell density of 25.7×10⁶ cells/ml (89% viability). The described minicap devices were attached to a peristaltic pump and the cell culture harvest was pumped through the devices. The filtrate volume was continuously recorded by means of a system of scales and a data recorder. No pre-use device flushing was necessary for the devices containing the sintered depth filter media as described in Example 3. The filter pressure drop was measured by means of an electronic pressure transducer. The filtration resistance profiles as a function of filter throughput were plotted (see Table 6 and FIG. 7) and these were compared to a conventional cellulose-based depth filter (MILLISTAK+® CE25 filter media) which was tested as a control sample. Several observations can be made from the results of this experiment. (1) No significant increase in system resistance was observed for the filter media of example ID 6-6. Increased PE binder loadings gave increased filter retention, as shown by the higher filter resistance profiles for example IDs 6-2 through 6-5. (2) The filter resistance profiles for example IDs 6-4 and 6-5 were similar. These results indicate that the sintered depth filter media in example ID 3-7 was not significantly degraded by the gamma radiation dose of 40-60 kGy. (3) The observed filter resistance profiles and retention characteristics are comparable to a conventional cellulose-based depth filtration media (MILLISTAK+® CE, MilliporeSigma, Burlington, MA) in harvest clarification unit operations.

TABLE 6

| | | | Filter | | Final |
|---|---|---|---|---|---|
| Example | | PE:PS | area | Flux | through- |
| ID | Filter Media | ratio | (cm²) | (LMH) | put (L/m²) |
| 6-1 | CE25 (Millistak+ ® | n/a | 23 | 133 | 163 |
| | benchmark) | | | | |
| 6-2 | example 3-4 | 7:3 | 23 | 146 | 169 |
| 6-3 | example 3-5 | 6:4 | 23 | 143 | 165 |
| 6-4 | example 3-7 | 1:1 | 23 | 141 | 157 |
| 6-5 | example 3-7 | 1:1 | 23 | 146 | 176 |
| | (40-60 kGy gamma dose) | | | | |
| 6-6 | example 3-3 | 3:7 | 23 | 133 | 163 |

### Example 7

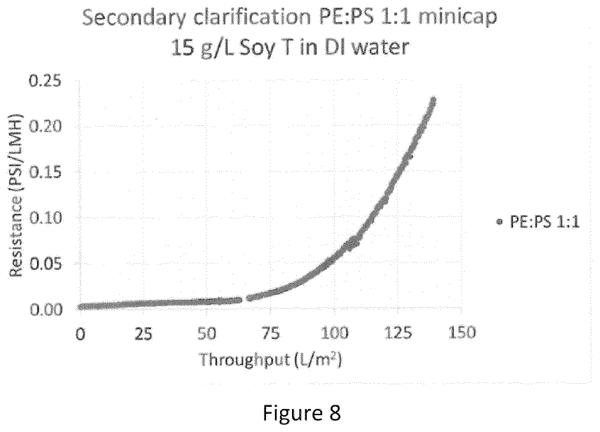

Clarification of model feedstream (Peptone HY-SOY® T). A 23 cm² minicap test device containing the sintered depth filter media described in example ID 3-2 was provided. The filter test device was flushed with 100 L/m² of Milli-Q water at a flow rate of 600 LMH. The filter test device was challenged with a model feedstream comprising a 15 g/L suspension of Peptone HY-SOY® T (P6463, MilliporeSigma) in DI (distilled, deionized) water. The turbidity of the model feed solution was measured to be 179 NTU at a 10×dilution. This model feedstream was chosen to approximate the depth filter plugging characteristics encountered in a typical secondary clarification application. Secondary clarification steps are generally employed for the removal of fine particles or turbidity reduction in selected applications including postcentrifuge, post-perfusion bioreactor, post-cell retention device, post-primary depth filtration, and post-protein A for certain downstream intermediates. The described minicap device was attached to a peristaltic pump and the Peptone HY-SOY® T feedstream was pumped through the device and the filtrate volume was continuously recorded by means of a scale and a data recorder. The filter pressure drop was measured by means of an electronic pressure transducer. The filtration resistance profile as a function of filter throughput was plotted. See Table 7 and FIG. 8. Several observations can be made from the results of this experiment. The observed filter resistance profile and retention characteristics (filtrate turbidity reduction) are comparable to the performance of a conventional single-layer diatomaceous earth-based depth filtration media grade (MILLISTAK+® DE type filter media, MilliporeSigma, Burlington, MA) in secondary clarification unit operations.

TABLE 7

| | | | Filter | | Final | |
|---|---|---|---|---|---|---|
| Example | Filter | PE:PS | area | Flux | throughput | Pool |
| ID | Media | ratio | (cm²) | (LMH) | (L/m²) | NTU |
| 7-1 | example 3-2 | 1:1 | 23 | 134 | 126 | 343 |

### Example 8

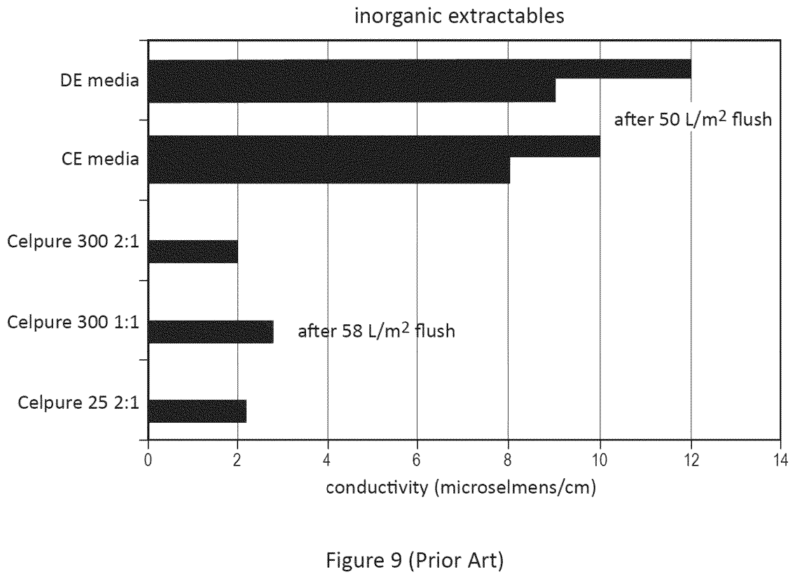

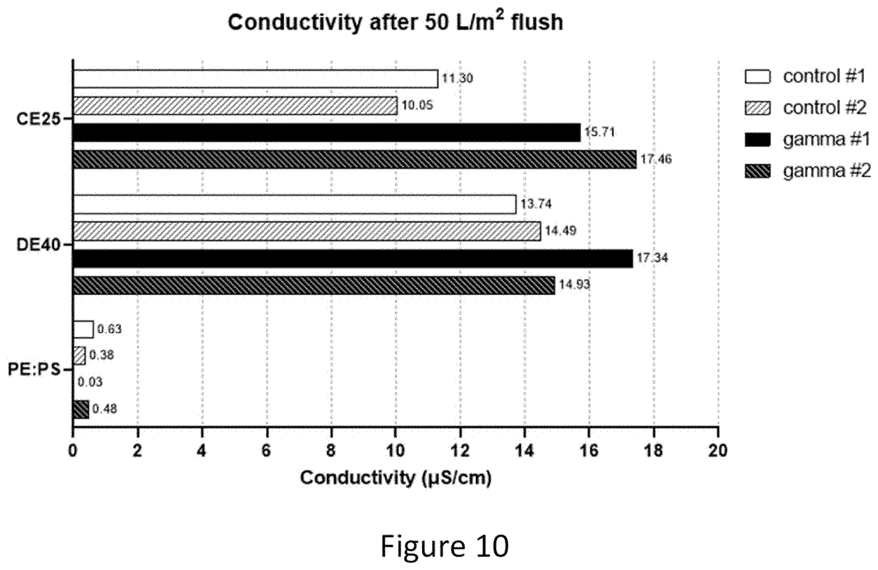

Commercial prior art media (e.g., DE and CE media) demonstrate high inorganic extractables (U.S. Pat. No. 7,673,757; the '757 patent, which is incorporated herein in its entirety). As a result, a high filtrate conductivity value of 8-12 μS/cm was measured after a 50 L/m² flush. The media described in the '757 patent show a decreased inorganic extractables content, and the filtrate conductivity value is reduced to 2 μS/cm after a 58 L/m² flush. See, prior art reference figure, FIG. 9, where the media of the '757 patent are denoted by the name “Celpure.” Based on the data provided in the '757 patent, one skilled in the art would recognize that pre-use flush volumes of greater than 50 L/m² will be required to use the CE or DE filter media. However, while reduced pre-use flush volumes of at least 58 L/m² would be sufficient for the media of the '757 patent, the required flush volumes are still considered unacceptable.

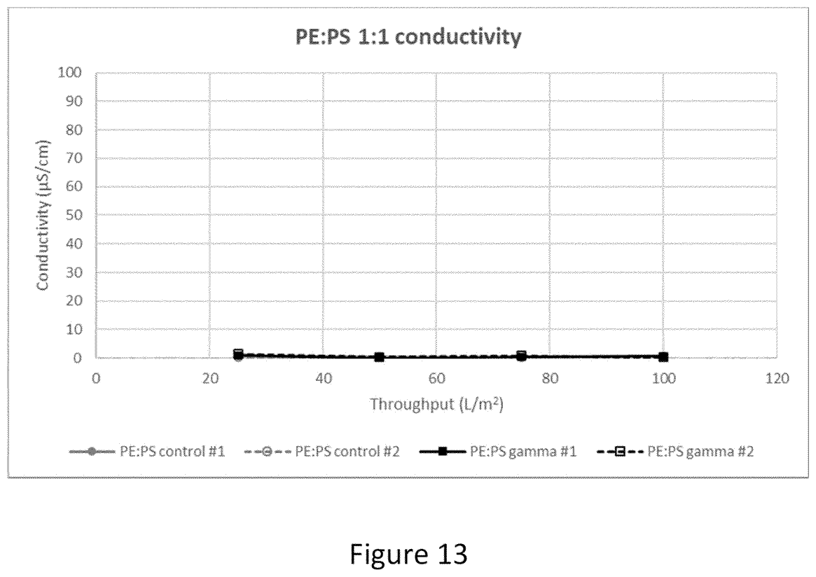

In contrast, the PE: PS 1:1 filter media described in our specification requires greatly reduced or no flush volumes prior to use as there are very low inorganic extractables from this filter material. The filtrate conductivity value is <0.5 μS/cm after a 25 L/m² flush (see, FIG. 13). One skilled in the art, armed with the teachings of this specification, would recognize that a filter media with such low inorganic extractables would not require a pre-use flush.

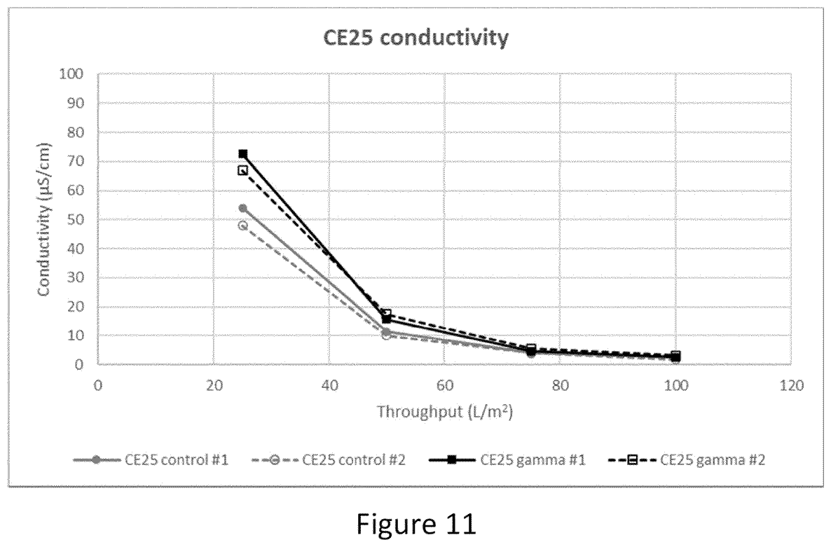

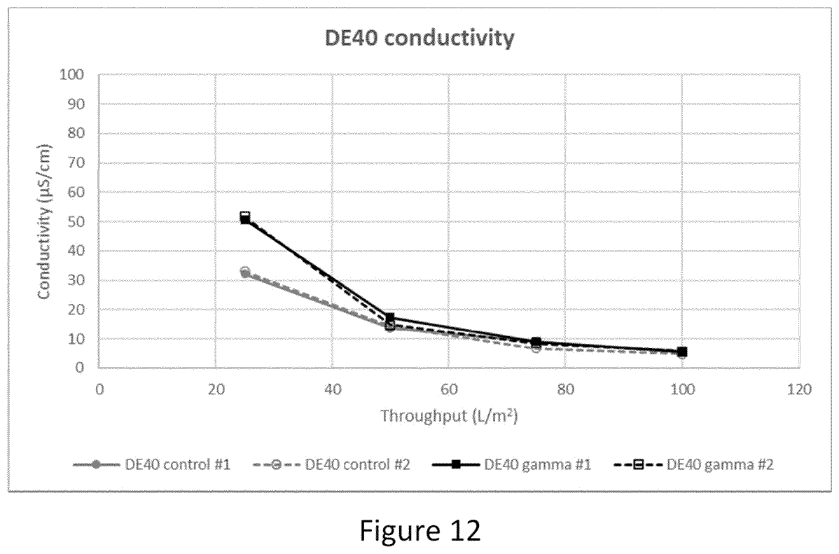

In a subsequent study, samples of CE media, DE media, and the PE: PS 1:1 filter media were exposed to a sterilizing dose of gamma radiation (25-40 kGy). While both the CE and DE media show an increase in filtrate conductivity values after gamma irradiation (see, FIGS. 11 and 12, respectively), no increase was observed for the PE: PS 1:1 material described in our specification (see, FIG. 13). Furthermore, filtrate conductivity values for the PE: PS 1:1 filter media were observed to be 50-100 times lower than the CE and DE filter media samples tested.

### Example 9

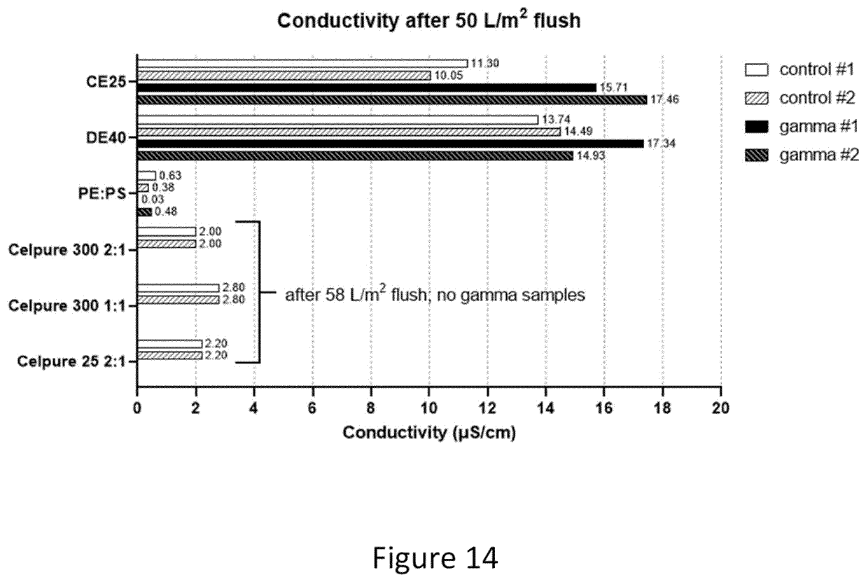

In this prophetic example, the composite materials of the type described in Example 3 of the '757 patent are flushed with clean deionized water (MILLIQ®, MilliporeSigma, Burlington, MA) and the conductivity of the effluent after a prescribed flush volume of 58 L/m² is measured. Conductivity values are taken to represent the level of soluble metals present in the filter media. FIG. 14 shows the conductivity values obtained for various Celpure/polyethylene composite material samples of the '757 patent relative to the values measured for the PE: PS 1:1 material of the present invention (see, FIG. 13). Conductivity data recorded for MILLISTAK+® CE25 (FIG. 11) and DE40 depth filter media (FIG. 12) are also provided for comparison. The effluent conductivity data for the Celpure/polyethylene composite material samples of the '757 patent shows a level of about 2 μS/cm after a flush of 58 L/m². Since the effluent conductivity values are representative of the level of soluble metals present in the filter media, these values are not expected to increase or decrease for the prior art Celpure/polyethylene composites after exposure to gamma radiation. As described at page 7, column 2, line 14 the '757 patent: “the extractables that contribute to conductivity (inorganics) do not solely or predominately derive from the diatomaceous earth . . . indeed, the composite materials of the present invention, devoid of cellulose and thermoset binder, result in a reduction in effluent conductivity of 75-90%, compared to conventional cellulosic media.” However, while the effluent conductivity is substantially reduced with the filter materials of the '757 patent, the metallic extractables contribution is not eliminated from the diatomaceous earth filter aid used in the composite materials described in the reference. In contrast, with the PE: PS 1:1 filter media of the present invention the effluent conductivity is further reduced by up to 95% compared to conventional cellulosic media such as DE40 and is several times lower as compared to the materials of the '757 patent. Depth filter materials such as the PE: PS 1:1 filter media described in the present invention would not “require pre-use flushing to reduce the level of organic or inorganic contaminants to acceptable levels prior to use” (the '757 patent, column 1, line 53) as is required with the prior art filter materials including the filter media of the '757 patent.

We claim:

1. A depth filter comprising;
a) a sintered depth filter media comprising a thermally fused mixture of polyethylene binder, and
b) one or more adsorbents selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene.

2. The depth filter of claim 1, wherein said depth filter media is significantly homogeneous.

3. The depth filter of claim 1, wherein said depth filter is from about 1 mm—about 4 mm thick.

4. The depth filter of claim 3, wherein said depth filter is from about 2 mm—about 3 mm thick.

5. The depth filter of claim 1, wherein wet-strength binder resins are specifically excluded.

6. The depth filter of claim 1, wherein said ratio of polyethylene to adsorbent is from about 1:10 to about 10:1.

7. The depth filter of claim 1, wherein said ratio of polyethylene to adsorbent is from about 1:5 to about 5:1.

8. The depth filter of claim 1, wherein said ratio of polyethylene to adsorbent is from about 1:2 to about 2:1.

9. The depth filter of claim 1, wherein said ratio of polyethylene to adsorbent is about 1:1.

10. The depth filter of claim 1, wherein the size of said polystyrene adsorbent is from about 10 μm to about 120 μm in diameter.

11. The depth filter of claim 1, wherein the size of the polystyrene adsorbent is from about 30 μm to 100 μm in diameter.

12. The depth filter of claim 1, wherein the size of the polystyrene absorbent is from about 50 μm to 80 μm in diameter.

13. The depth filter of claim 1, wherein the size of the polystyrene absorbent is from about 60 μm to 70 μm in diameter.

14. The depth filter of claim 1, wherein the size of the polystyrene absorbent is about 63 μm in diameter.

15. A method of making a sintered depth filter media, the method comprising mixing a polyethylene binder with an adsorbent selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene to make a mixture, spreading the mixture to a substantially uniform thickness and heating the mixture at about 165° C. for about 60 minutes.

16. The sintered depth filter media made by the method of claim 15, wherein said depth filter media is significantly homogeneous.

17. The sintered depth filter media made by the method of claim 15, wherein said depth filter is from about 1 mm—about 4 mm thick.

18. The sintered depth filter media made by the method of claim 17, wherein said depth filter is from about 2 mm—about 3 mm thick.

19. The sintered depth filter media made by the method of claim 15, wherein wet-strength binder resins are specifically excluded.

20. The sintered depth filter media made by the method of claim 15, wherein said ratio of polyethylene to adsorbent is from about 1:10 to about 10:1.

21. The sintered depth filter media made by the method of claim 15, wherein said ratio of polyethylene to adsorbent is from about 1:5 to about 5:1.

22. The sintered depth filter media made by the method of claim 15, wherein said ratio of polyethylene to adsorbent is from about 1:2 to about 2:1.

23. The sintered depth filter media made by the method of claim 15, wherein said ratio of polyethylene to adsorbent is about 1:1.

24. The sintered depth filter media made by the method of claim 15, wherein the size of said adsorbent is from about 10 μm to about 120 μm in diameter.

25. The sintered depth filter media made by the method of claim 24, wherein the size of the adsorbent is from about 30 μm to 100 μm in diameter.

26. The sintered depth filter media made by the method of claim 25, wherein the size of the adsorbent is from about 50 μm to 80 μm in diameter.

27. The sintered depth filter media made by the method of claim 26, wherein the size of the adsorbent is from about 60 μm to 70 μm in diameter.

28. The sintered depth filter media made by the method of claim 27, wherein the size of the adsorbent is about 63 μm in diameter.

29. A method of clarifying a feedstream, the feed stream containing a target protein, the method comprising:
a) providing: a depth filter comprising a sintered depth filter media, said depth filter media comprising a polyethylene binder with an adsorbent selected from the group consisting of PrAOH, PrCH, MB1/1h, polystyrene adsorbent, poly(4-vinylbenzene) 2% cross-linked with divinylbenzene, poly(4-vinylpyridine-co-ethylvinylbenzene) 25% cross-linked with divinylbenzene;
b) contacting the depth filter with the feed stream such that the target protein passes through the depth filter and is retained in the permeate and contaminants are adsorbed by the depth filter, such that the concentration of target protein to contaminant in the permeate is increased by at least 20% as compared to the ratio in the feed stream.

30. The method of claim 29, wherein said concentration of target protein to contaminant in the permeate is increased by at least 50% as compared to the ratio in the feed stream.

31. The method of claim 29, wherein said concentration of target protein to contaminant in the permeate is increased by at least 100% as compared to the ratio in the feed stream.

32. The method of claim 29, wherein said depth filter media is significantly homogeneous.

33. The method of claim 29, wherein said depth filter is from about 1 mm—about 4 mm thick.

34. The method of claim 33, wherein said depth filter is from about 2 mm—about 3 mm thick.

35. The method of claim 29, wherein wet-strength binder resins are specifically excluded.

36. The method of claim 29, wherein said ratio of polyethylene to adsorbent is from about 1:10 to about 10:1.

37. The method of claim 29, wherein said ratio of polyethylene to adsorbent is from about 1:5 to about 5:1.

38. The method of claim 29, wherein said ratio of polyethylene to adsorbent is from about 1:2 to about 2:1.

39. The method of claim 29, wherein said ratio of polyethylene to adsorbent is about 1:1.

40. The method of claim 29, wherein the size of said adsorbent is from about 10 μm to about 120 μm in diameter.

41. The method of claim 40, wherein the size of the adsorbent is from about 30 μm to 100 μm in diameter.

42. The method of claim 41, wherein the size of the adsorbent is from about 50 μm to 80 μm in diameter.

43. The method of claim 42, wherein the size of the adsorbent is from about 60 μm to 70 μm in diameter.

44. The method of claim 43, wherein the size of the adsorbent is about 63 μm in diameter.
